(19) United States
(12) Patent Application Publication
(10) Pub. No.: US 2018/0311026 A1
(43) Pub. Date: Nov. 1, 2018

(54) MEDICAL DEVICES WITH ANTI-MIGRATION SURFACES AND METHODS OF PREPARING THE SAME

(71) Applicant: Boston Scientific Scimed, Inc., Maple Grove, MN (US)

(72) Inventor: Gerald Fredrickson, Westford, MA (US)

(21) Appl. No.: 15/964,761

(22) Filed: Apr. 27, 2018

Related U.S. Application Data
(60) Provisional application No. 62/491,631, filed on Apr. 28, 2017.

(51) Int. Cl. A61F 2/00; A61L 31/08

(52) U.S. Cl. CPC A61F 2/0077; A61F 2/848; A61L 31/08

(57) ABSTRACT

The present disclosure relates to the field of medical devices with anti-migration capabilities, such as anti-migration surfaces. Specifically, the present disclosure relates to methods for covalently bonding tissue-adhesive functional groups to the outer surface of a medical device to provide a medical device with an anti-migration surface.

### CROSS REFERENCE TO RELATED APPLICATIONS

The present application claims the benefit of priority under 35 U.S.C. § 119 to U.S. Provisional Patent Application Ser. No. 62/491,631, filed on Apr. 28, 2017, which is incorporated by reference in its entirety for all purposes.

### FIELD

The present disclosure relates to the field of medical devices with anti-migration capabilities. Specifically, the present disclosure relates to methods for covalently bonding a tissue-adhesive functional group to an outer surface of a medical device to provide a medical device with an anti-migration surface.

### BACKGROUND

Post-implant migration from the intended intraluminal location occurs, e.g., in up to 20 percent of stent delivery procedures, and may lead to a variety of medical complications, including with stents as an example lumen re-occlusion and the need to retrieve and/or replace the improperly positioned stent. Stent migration is especially problematic with polymeric or coated stents due, at least in part, to their low-friction outer surfaces and inherent resistance to tissue ingrowth. Conventional anti-migration stents include adhesive coatings and/or intermittently arranged surface features (e.g., hooks, barbs, flared ends, etc.) configured to engage the inner wall of a target body lumen. Adhesive coatings tend to degrade or erode over time, potentially resulting in stent migration and the possible release of harmful substances/compounds into the patient. Surface features that physically engage the inner wall of a body lumen tend to cause tissue irritation and/or trauma which may promote restenosis of an already unhealthy or otherwise compromised site.

Accordingly, various advantages may be realized by medical devices and methods which provide an effective anti-migration capability, such as an anti-migration surface, which includes a non-degradable/non-erodible tissue-adhesive functional group(s) covalently bonded to the outer surface of the medical device to limit or prevent intraluminal migration.

### SUMMARY

The present disclosure, in its various aspects, provides advantages in the medical field, such as the field of intraluminal devices (e.g., stents, etc.), for devices and methods that provide anti-migration functionality, such as devices with non-degradable/non-erodible anti-migration outer surfaces. In various embodiments, the outer surface of these medical devices may be covalently bonded to one or more biomimetic tissue-adhesive functional groups to limit or prevent intraluminal migration.

In one aspect, the present disclosure relates to a method of preparing a medical device with a tissue-adhesive surface, comprising reacting the medical device with an effective amount of a tissue-adhesive functional group, such that one or more hydroxyl groups on a surface of the medical device covalently bond with one or more binding groups of the tissue-adhesive functional group, thereby forming a medical device with a tissue-adhesive surface. The one or more binding groups may comprise silane. The tissue-adhesive functional group may comprise an aldehyde, including, for example, Acetoxymethyltriethoxysilane. The Acetoxymethyltriethoxysilane may be covalently bonded directly to the one or more hydroxyl groups on the surface of the medical device using silane chemistry. The medical device may comprise silicone. The hydroxyl groups may be a component of a silica filler of the silicone. The medical device may be formed entirely from silicone. Alternatively, the medical device may be at least partially coated (e.g., on and inner and/or outer surface thereof) with silicone. The method may further comprise, prior to the reacting step, generating hydroxyl groups on a surface of the medical device. The hydroxyl groups may be generated on a surface (e.g., inner and/or outer surface) of the silicone. The hydroxyl groups may be generated by a plasma discharge, corona discharge or NaOH soak.

In another aspect, the present disclosure relates to a method of preparing a medical device with a tissue-adhesive surface, comprising reacting the medical device bonded with the primer-terminated silane with an effective amount of a primer-terminated silane, such that one or more hydroxyl groups on a surface of the medical device covalently bond with one or more binding groups of the primer-terminated silane, and reacting the medical device with an effective amount of a tissue-adhesive functional group, such that one or more amine groups of the tissue-adhesive functional group covalently bond with one or more silane groups of the primer-terminated silane, thereby forming the medical device with a tissue-adhesive surface. The primer-terminated silane may comprise (3-Glycidoxypropyl)Trimethoxysilane. The tissue-adhesive functional group may comprise a catechol, including, for example (3,4-dihydroxyphenethylamine).

In yet another aspect, the present disclosure relates to a medical device, comprising an outer surface functionalized with a tissue-adhesive functional group. The tissue-adhesive functional group may include an aldehyde. In addition, or alternatively, the tissue-adhesive functional group may include a catechol. In addition, or alternatively, the tissue-adhesive functional group may include a blend of functional groups. The medical device may include, by way of non-limiting example, a stent. The medical device may be placed within a body lumen such that a tissue-adhesive surface of the medical device reacts with amines in the tissue wall of the body lumen, thereby securing the medical device within the body lumen.

### BRIEF DESCRIPTION OF THE DRAWINGS

The above and further advantages of the devices and methods of the present disclosure may be better understood by referring to the following description in conjunction with the accompanying drawings, in which:

FIG. 1 illustrates the steps involved in covalently bonding a tissue-adhesive functional group to the outer surface of a medical device, according to one embodiment of the present disclosure.

FIG. 2 illustrates the covalent bonding of a tissue-adhesive functional group to a silicone substrate by a silane chemistry reaction, according to one embodiment of the present disclosure.

FIG. 3 illustrates the generation of hydroxyl (OH) groups on the surface of a silicone substrate, according to one embodiment of the present disclosure.

FIG. 4 illustrates the steps involved in covalently bonding a tissue-adhesive functional group to the outer surface of a medical device, according to one embodiment of the present disclosure.

It is noted that the drawings are intended to depict only typical or exemplary embodiments of the disclosure. Accordingly, the drawings should not be considered as limiting the scope of the disclosure. The disclosure will now be described in greater detail with reference to the accompanying drawings.

### DETAILED DESCRIPTION

Before the present disclosure is described in further detail, it is to be understood that the disclosure is not limited to the particular embodiments described, as such may vary. It is also to be understood that the terminology used herein is for the purpose of describing particular embodiments only, and is not intended to be limiting beyond the scope of the appended claims. Unless defined otherwise, all technical terms used herein have the same meaning as commonly understood by one of ordinary skill in the art to which the disclosure belongs. Finally, although embodiments of the present disclosure are described with specific reference to silicone-containing stents that include tissue-adhesive functional groups covalently bonded to the outer surface thereof, the systems and methods disclosed herein may be used to functionalize the outer and/or inner surface of a variety of silicone, polymeric and/or metallic medical devices (e.g., catheters, grafts, guidewires, aneurysm coils, enteral feeding devices, cardiac lead sensors, etc.), including coatings, liners, covers therefore, and the like, to prevent or inhibit migration within a variety of body lumens, body passages, organs, spaces between organs, and the like.

As used herein, the singular forms “a,” “an,” and “the” are intended to include the plural forms as well, unless the context clearly indicates otherwise. It will be further understood that the terms “comprises” and/or “comprising,” or “includes” and/or “including” when used herein, specify the presence of stated features, regions, steps elements and/or components, but do not preclude the presence or addition of one or more other features, regions, integers, steps, operations, elements, components and/or groups thereof.

The present disclosure generally relates to medical devices (e.g., stent) comprising a hydroxyl-containing surface covalently bonded to the functional group of a biomimetic adhesive. When the medical device is disposed within a body lumen, the covalently bonded adhesive functional group may provide an anti-migration surface by reacting with (e.g., bonding to) amine groups of the apposed tissue wall of the body lumen. Examples of biologically derived adhesives which may find beneficial use in the devices and methods of the present disclosure, include, but are not limited to, fibrin sealants, aldehydes (e.g., gelatin-resorcin aldehydes, protein-aldehydes, etc.) collagen-based adhesives, polysaccharide-based adhesives, epoxies, catechol groups, thiol groups, mussel adhesive proteins and various biologically inspired or biomimetic glues.

A variety of methods in accordance with the present disclosure are available for covalently attaching a tissue-adhesive functional group to the silicone, polymeric or metallic surface, liner, cover or coating of a medical device. In one embodiment, a reactive aldehyde functional group may be covalently attached to the surface of a medical device using silane chemistry.

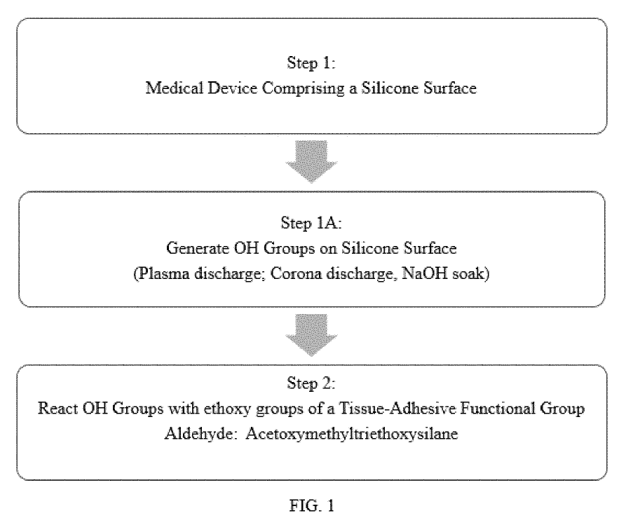

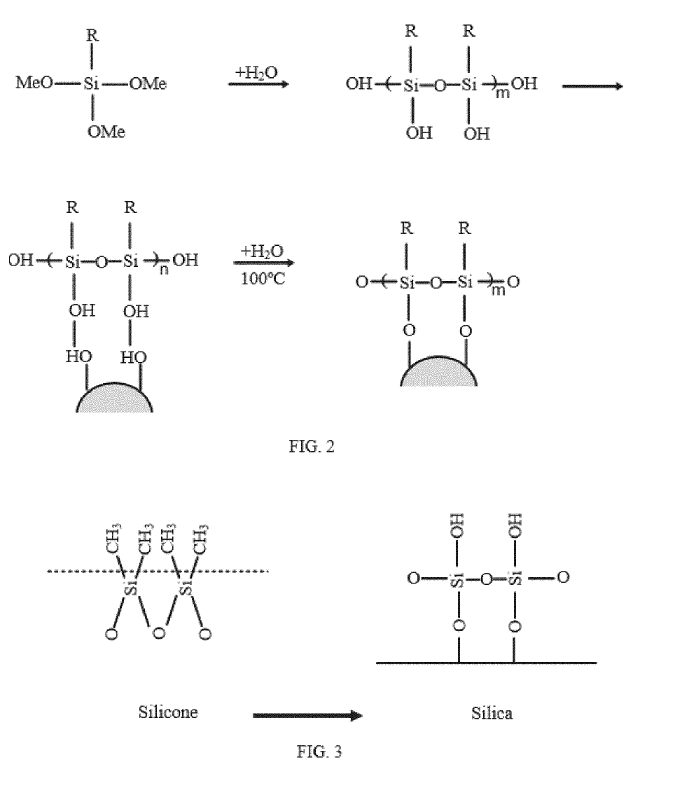

For example, referring to FIG. 1, a tissue-adhesive surface may be prepared (e.g., functionalized) by directly reacting the silicone surface of a medical device with an effective amount of an active component (e.g., tissue-adhesive functional group) comprising Acetoxymethyltriethoxysilane under conditions such that the ethoxy groups react with hydroxyl groups on the silicone surface of the medical device to leave the aldehyde groups free to react with amines on the tissue surface. More specifically, referring to FIG. 2, the methoxy groups (OMe) of the Acetoxymethyltriethoxysilane may be hydrolyzed by water to produce methanol and leave (e.g., evaporate from) the silicone and provide a reaction site (e.g., hydroxyl groups) to react with the hydroxyl groups on the silicone surface of the medical device. The hydroxyl groups of the Acetoxymethyltrihydroxysilane may then react with the hydroxyl groups present on the silicone surface of the medical device in an exothermic reaction that produces water and covalently bonds the Acetoxymethyltrihydroxysilane to the silicone surface of the device by a silicone-oxygen bond, and leaves the aldehyde group (R) free to react with amines on the tissue surface. In one embodiment, a medical device may include sufficient hydroxyl groups to support the silane chemistry reaction due to the presence of silica filler embedded on or within the silicone surface. Referring to FIG. 3, in addition, or alternatively, additional hydroxyl groups may be generated on the silicone surface of the medical device using, e.g., plasma discharge, corona discharge and/or NaOH soak, as are commonly known in the art.

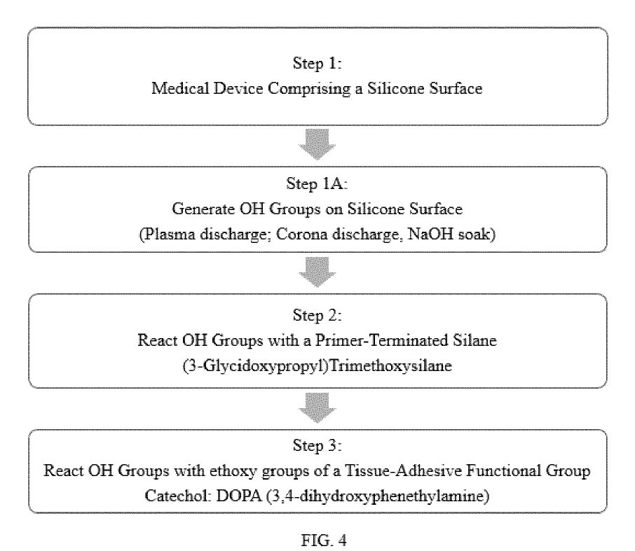

In another embodiment, a reactive catechol functional group may be covalently attached to the surface of a medical device using silane chemistry. For example, referring to FIG. 4, a tissue adhesive surface may be prepared by reacting the surface of a medical device with an effective amount of an amine reactive group, including, e.g., (3-Glycidoxypropyl)Trimethoxysilane, carboxylic acids, esters, ketones and aldehydes, under conditions such that the primer-terminated silane reacts with the hydroxyl groups present on the silicone surface of the medical device. This intermediate may provide a functionalized substrate that includes terminal silanes which may be reacted with an effective amount of DOPA (e.g., 3,4-dihydroxyphenethylamine) under conditions such that the terminal silane reacts with the amine group of the DOPA. As above, additional hydroxyl (OH) groups may be generated on the silicone surface of the medical device using, e.g., plasma discharge, corona discharge and/or NaOH soak.

In certain embodiments, it may be preferable for only the outer surface of the medical device to be functionalized with the tissue-adhesive functional groups. Accordingly, in one embodiment, the entire surface (e.g., inner and outer surfaces) of the medical device may undergo plasma discharge, corona discharge and/or NaOH soak to provide the requisite hydroxyl groups to facilitate the silane chemistry reaction, but the aldehyde or catechol functional group is only reacted with the outer surface of the medical device.

Although the present disclosure provides methods for attaching tissue-adhesive aldehyde or catechol groups to the outer surface of a medical device, in various embodiments, a combination (e.g., blend) of these or other tissue-adhesive functional groups may be covalently bonded to the medical device. For example, an outer surface of a medical device may include a functional layer of, e.g., epoxy silane or aldehyde silane, which is then partially reacted with a limited amount of a catechol group, thereby providing an outer surface that includes some pendant catechol groups and some aldehyde (or epoxy) groups.

In use and by way of example, a medical device (e.g., stent) of the present disclosure may be advanced into a body lumen such that the outer surface of the medical device is placed in radial (e.g., 360 degree) contact with the tissue wall of the body lumen. The tissue-adhesive functional groups covalently attached to the outer surface of the medical device may then react with (e.g., bond to) the amine groups present in the tissue wall of the body lumen to secure, affix or adhere the medical device with respect to the body lumen such that migration limited or prevented. For example, the tissue-adhesive functional groups may covalently attach to the outer surface of the medical device within minutes to hours following implantation. In various embodiments, the presence of non-erodible/non-degradable tissue-adhesive functional groups along the entire outer surface of the medical device may limit or prevent migration of the medical device from the intended point of deployment to an extent that is unachievable using conventional adhesive coatings and/or intermittent surface features. Moreover, since tissue-adhesion only occurs at a surface of the medical device, less force is required to remove or reposition the medical device than with medical devices that support tissue ingrowth. This reduction in removal or repositioning force may result in less trauma to the tissue wall of the body lumen.

In one embodiment, the medical devices disclosed herein may include a metallic or polymeric stent formed from a variety of woven or braided materials, and which are partially or completely coated, covered and/or or embedded within a layer of silicone. By way of non-limiting example, stents may include laser cut stents which may or may not change in length (e.g., shorten) as the stent moves from the first configuration to the second configuration. In addition, or alternatively, the stents in various configurations may be self-expanding or expandable such as balloon-expandable. Alternatively, the medical devices disclosed herein may be formed entirely from silicone (or other suitable polymeric material) without an underlying woven or braided framework.

Although the present disclosure is described with specific reference to silane chemistry reactions performed on silicone substrates, in various embodiments, these silane chemistry reactions may be performed on a variety of suitable polymeric (e.g., plastic) and/or metallic substrates that include, or may be modified to include, free hydroxyl (OH) groups.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A method of preparing a medical device with a tissue-adhesive surface, comprising:
reacting the medical device with an effective amount of a tissue-adhesive functional group, such that one or more hydroxyl groups on a surface of the medical device covalently bond with one or more binding groups of the tissue-adhesive functional group, thereby forming the medical device with a tissue-adhesive surface.

2. The method of claim 1, wherein the one or more binding groups comprise silane.

3. The method of claim 1, wherein the tissue-adhesive functional group comprises an aldehyde.

4. The method of claim 3, wherein the aldehyde includes Acetoxymethyltriethoxysilane.

5. The method of claim 4, wherein the Acetoxymethyltriethoxysilane is covalently bonded directly to the one or more hydroxyl groups on the surface of the medical device using silane chemistry.

6. The method of claim 1, wherein the medical device comprises silicone.

7. The method of claim 6, wherein the hydroxyl groups are a component of a silica filler of the silicone.

8. The method of claim 1, wherein the medical device is formed entirely from silicone.

9. The method of claim 6, further comprising, prior to the reacting step, generating hydroxyl groups on a surface of the medical device.

10. The method of claim 9, wherein the hydroxyl groups are generated on a surface of the silicone.

11. The method of claim 9, wherein the hydroxyl groups are generated by a plasma discharge, corona discharge or NaOH soak.

12. A method of preparing a medical device with a tissue-adhesive surface, comprising:
reacting the medical device bonded with the primer-terminated silane with an effective amount of a primer-terminated silane, such that one or more hydroxyl groups on a surface of the medical device covalently bond with one or more binding groups of the primer-terminated silane; and
reacting the medical device with an effective amount of a tissue-adhesive functional group, such that one or more amine groups of the tissue-adhesive functional group covalently bond with one or more silane groups of the primer-terminated silane, thereby forming the medical device with a tissue-adhesive surface.

13. The method of claim 12, wherein the primer-terminated silane comprises (3-Glycidoxypropyl)Trimethoxysilane.

14. The method of claim 12, wherein the tissue-adhesive functional group comprises a catechol.

15. The method of claim 14, wherein the catechol includes (3,4-dihydroxyphenethylamine).

16. A medical device, comprising an outer surface functionalized with a tissue-adhesive functional group.

17. The medical device of claim 16, wherein the tissue-adhesive functional group includes an aldehyde.

18. The medical device of claim 16, wherein the tissue-adhesive functional group includes a catechol.

19. The medical device of claim 16, wherein the tissue-adhesive functional group includes a blend of functional groups.

20. The medical device of claim 16, wherein the medical device is a stent.
